(19) United States
(12) Patent Application Publication
(10) Pub. No.: US 2019/0365852 A1
(43) Pub. Date: Dec. 5, 2019

(54) RECOMBINANT BACULOVIRUSES AND USES THEREOF

(71) Applicants: Academia Sinica, Taipei (TW); Chung Yuan Christian University, Taoyuan City (TW); National Defense Medical Center, Taipei City (TW)

(72) Inventors: Yu-Chan CHAO, Taipei City (TW); Nenavath Gopal NAIK, Taipei City (TW); Tzong-Yuan WU, Taoyuan City (TW); Chang-Chi LIN, New Taipei City (TW); Szu-Cheng KUO, New Taipei City (TW)

(73) Assignees: Academia Sinica, Taipei (TW); Chung Yuan Christian University, Taoyuan City (TW); National Defense Medical Center, Taipei City (TW)

(21) Appl. No.: 15/992,178

(22) Filed: May 30, 2018

(51) Int. Cl. A61K 38/17; A01N 63/00; C07K 14/005; C07K 14/47; C12N 15/65; C12N 15/79

(52) U.S. Cl. CPC A61K 38/1729; A01N 63/00; C07K 14/005; C07K 14/4723; C12N 2310/141; C12N 15/79; C12N 2799/026; C12N 2799/04; C12N 15/65

(57) ABSTRACT

Disclosed herein are recombinant baculoviruses suitable for introducing an exogenous gene into a pest insect, particularly, disease-transmitting mosquitos. The recombinant baculovirus is characterized in having a promotor that is any of a HzNV-1 viral early expressing gene pag1, a ceropin gene b1, a defensin gene a4, or hp70 gene; and an exogenous gene operably linked thereto the promoter. Also disclosed herein is a method of introducing an exogenous gene into a pest insect. The method includes transducing the pest insect with a recombinant baculovirus without suppressing the production of microRNAs (miRNAs) in the pest insect, wherein the recombinant baculovirus comprises a promoter of pag1, cecropin b1, defensin gene a, or hp70.

### BACKGROUND OF THE INVENTION

### 1. Field of the Invention

The present disclosure relates to modified vectors for gene delivery. More particularly, the disclosure relates to a recombinant baculovirus for delivering an exogenous gene into a mosquito, particularly, a disease-transmitting mosquito.

### 2. Description of Related Art

Aedes aegypti (the yellow fever mosquito) and A. albopictus (Asian tiger mosquito or forest mosquito) are responsible for the transmission of various potentially fatal viruses in humans including dengue, chikungunya, zika, yellow fever, and Mayaro, as well as several filarial nematodes such as Dirofilaria immitis and other diseases. Culex tritaeniorhynchus is the main vector of Japanese encephalitis, and Anopheles sinensis transmits malaria and lymphatic filariasis. Despite continuous study of mosquito gene regulation and efforts to prevent mosquito-borne diseases, lack of efficient and flexible gene delivery approaches hinder investigations into virus/host interactions and mosquito biology. An efficient gene delivery system into cells, larvae and different organs of adults and across different mosquito species would obviously be an indispensable tool for such studies and have many other crucial applications in biological research.

In view of the above, there exist in this art a need of a tool and/or methods for delivering genes into mosquitos, so as to better study and control mosquitos from transmitting dangerous virus to human and/or live stock.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

The present disclosure relates in general to modified vectors and uses thereof. The modified vectors are suitable for introducing exogenous genes into mosquitos, particularly, disease-transmitting mosquitos such as Aedes aegypti, A. albopictus, Culex tritaeniorhynchus, and Anopheles sinensis.

Accordingly, the first aspect of the present disclosure aims at providing a recombinant baculovirus capable of delivering an exogenous gene into a mosquito. The recombinant baculovirus is characterized in having a HzNV-1 viral early expressing gene pag1, a ceropin gene b1, a defensin gene a4, or a gene of heat shock protein 70 (hp70) as a promoter, and the exogenous gene operably linked to the promoter.

According to optional embodiments of the present disclosure, the recombinant baculovirus may further comprise a reporter gene operably linked to the promotor and the exogeneous gene and encodes a reporter protein for easy tracking and/or monitoring the expression. Examples of the reporter protein include, but are not limited to, green fluorescence protein (GFP), enhanced green fluorescence protein (EGFP), Discosoma sp. red fluorescent protein (DsRed), blue fluorescence protein (BFP), enhanced yellow fluorescent proteins (EYFP), Anemonia majano fluorescent protein (amFP), Zoanthus fluorescent protein FP), Discosoma fluorescent protein (dsFP), and Clavularia fluorescent protein (cFP). In one preferred embodiment, the reporter protein is EGFP.

Examples of baculovirus suitable for use in the present disclosure include, and are not limited to, Autographa californica multiple nucleopolyhedrovirus (AcMNPV), Anagrapha falclfera MNPV (AfMNPV), Anticarsia gemmatalis MNPV (AgMNPV), Bombyx mori MNPV (BmMNPV), Buzura suppressaria single nucleopolyhedrovirus (BsSNPV), Helicoverpa armigera SNPV (HaSNPV), Helicoverpa zea SNPV (HzSNPV), Lymantria dispar MNPV (LdMNPV), Orgyia pseudotsugata MNPV (OpMNPV), Spodoptera frugiperda MNPV (SfMNPV), Spodoptera exigua MNPV (SeMNPV), and Trichoplusia ni MNPVMNPV). According to one preferred embodiment, the recombinant baculovirus is a recombinant AcMNPV.

The second aspect of the present disclosure is directed to a method of delivering an exogenous gene into a mosquito via use of the present recombinant baculovirus. The method includes, transducing the mosquito with the present recombinant baculovirus without suppressing the production of microRNAs (miRNAs) in the mosquito. The present recombinant baculovirus is characterized in having a promoter that is any of a HzNV-1 viral early expressing gene pag1, a ceropin gene b1, a defensin gene a4, or a heat shock protein 70 gene; and the exogenous gene operably linked to the promoter.

According to embodiments of the present disclosure, the suppressing of the production of miRNAs in the mosquito is achieved by use of an agent that leads to suppressing the expression of Drosha, Dicer, Toll-like receptor 2, STAT1, STAT6, interleukin 7R, or interleukin 1A.

According to embodiments of the present disclosure, the mosquito is Aedes aegypti or A. albopictus.

According to embodiments of the present disclosure, the mosquito is a cell, a larvae or an adult.

Examples of baculovirus suitable for use in the present disclosure include, and are not limited to, Autographa californica multiple nucleopolyhedrovirus (AcMNPV), Anagrapha falclfera MNPV (AfMNPV), Anticarsia gemmatalis MNPV (AgMNPV), Bombyx mori MNPV (BmMNPV), Buzura suppressaria single nucleopolyhedrovirus (BsSNPV), Helicoverpa armigera SNPV (HaSNPV), Helicoverpa zea SNPV (HzSNPV), Lymantria dispar MNPV (LdMNPV), Orgyia pseudotsugata MNPV (OpMNPV), Spodoptera frugiperda MNPV (SfMNPV), Spodoptera exigua MNPV (SeMNPV), and Trichoplusia ni MNPVMNPV). According to one preferred embodiment, the recombinant baculovirus is a recombinant AcMNPV.

According to optional embodiments of the present disclosure, the recombinant baculovirus further comprises a reporter gene operably linked to the promotor and the exogeneous gene and encodes a reporter protein, which may be any of green fluorescence protein (GFP), enhanced green fluorescence protein (EGFP), Discosoma sp. red fluorescent protein (DsRed), blue fluorescence protein (BFP), enhanced yellow fluorescent proteins (EYFP), Anemonia majano fluorescent protein (amFP), Zoanthus fluorescent protein (zFP), Discosoma fluorescent protein (dsFP), or Clavularia fluorescent protein (cFP).

According to embodiments of the present disclosure, the recombinant baculovirus does not replicate in the mosquito.

According to embodiments of the present disclosure, the exogenous gene is successfully expressed in the head, proboscis, leg, haltere, midgut malphigian tubules, ovary, crop, and fat body of the adult mosquito.

Many of the attendant features and advantages of the present disclosure will becomes better understood with reference to the following detailed description considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIG. 1: Baculovirus transduction into mosquito C6/36 cells. (A) Schematic representation of a composite expression vector for generating recombinant baculoviruses. The composite transfer vector contains the sv40 and pag1 promoters, which are designed to express mCherry in mammalian and insect cells, respectively. (B) Dose-dependent entry of baculovirus. C6/36 cells were transduced with different concentrations (MOI=1, 10, and 100) of recombinant vABspmC baculovirus. (C) Gating of mCherry-positive cells by flow cytometry, shown for one representative biological replicate. (D) Quantification of mCherry-positive cells to indicate transduction efficiency. (E) Persistent baculovirus-mediated gene expression. C6/36 cells were transduced with baculovirus at MOI=1 and expression of fluorescent protein was analyzed over several days. The mCherry fluorescence images at various time-points were taken by fluorescence microscopy. (F) GP64-mediated entry of baculovirus. C6/36 cells were treated with a baculovirus-antibody mixture (neutralizing or non-neutralizing antibodies against GP64 protein). The mCherry fluorescence images were taken at 48 h post-transduction in both panels B and H. All the experiments were done in three biological replicates.

FIG. 2: Replication analysis of baculovirus in C6/36 cells. (A) mCherry fluorescence images of baculovirus transduction. C6/36 cells were transduced with vABspmC at MOI=10 and mCherry fluorescence images were captured at various time-points as shown. (B) Baculovirus entry efficiency. Baculovirus was transduced into C6/36 cells or infected with MOI=10 and MOI=50 for 2 h into Sf21 cells before relative entry efficiencies were quantified by qPCR. (C) Replication efficiency of baculovirus. C6/36 or Sf21 cells were transduced or infected with MOI=10, harvested at various time-points, and intracellular viral DNA was quantified by qPCR. Amounts of viral DNA at all time-points were normalized against an internal gene control—GAPDH for Sf21 or Actin for C6/36—and further normalized against viral DNA copy number after 2 h post-transduction. The experiments were performed independently three times.

FIG. 3: Efficiency of baculovirus-incorporated promoters in C6/36 cells. (A) Schematic representation of transfer vectors used to generate the recombinant baculoviruses expressing EGFP driven by several baculovirus, mammalian viral, and mosquito host promoters from the following genes: pag1, a HzNV-1 viral early expressing gene; p10, baculovirus late gene; cmv, cytomegalovirus; sv40, simian virus 40; lir, chimeric internal ribosome entry site (IRES) of RhPV virus and EV71 virus; b1, cecropin b1 gene; a4, defensin a4 gene; and pub, polyubiquitin gene. (B) Fluorescence images. C6/36 cells were transduced with recombinant viruses expressing EGFP at an MOI=1 or transfected with 500 ng of the recombinant plasmid DNA constructs used to generate recombinant baculoviruses. The images were taken by fluorescence microscopy at 48 h post-transduction. (C) Flow cytometry analysis of EGFP fluorescence driven by the promoters of interest. Recombinant baculovirus-transduced or plasmid DNA-transfected C6/36 cells were collected after 48 h and mean EGFP fluorescence intensities were measured by flow cytometry to determine promoter efficiency. Data represent the average of three biological replicates.

FIG. 4: Effect of hr1hsp70 promoter in baculovirus mediated EGFP expression in mosquito C6/36 cells. C6/36 cells were transduced with recombinant baculoviruses, vABhh-EG at MOI=1. The transduced C6/36 cells were collected after 48 h and the mean florescence intensities of EGFP were measured by flow cytometry determining the promoter's strength. The experiment was performed in triplicate, and the data shown is one representative experiment. Asterisks (p<0.0005***) indicate results significantly different from the mock and vABhh-EG respectively.

FIG. 5: Analysis of in vivo baculovirus transduction of mosquitoes. (A) Transduction of baculovirus into mosquito larvae. Four different mosquito species (A. aegypti, A. albopictus, C. tritaeniorhynchus and A. sinensis) were microinjected with 1×10⁵ PFU of vABb1EG-irEG baculovirus. EGFP expression was visualized by fluorescence microscopy at 2 days post-transduction. (B) Dose-dependent expression of a baculovirus-mediated transgene in adult mosquitoes. Four different mosquito species were microinjected intrathoracically with several doses (1×10³, 10⁴ or 10⁵ PFU) of vABb1EG-irEG baculovirus. EGFP expression was visualized by fluorescence microscopy at 6 days post-transduction. (C) Transduction of baculovirus into adult mosquitoes. Four different mosquito species were microinjected intrathoracically with 1×10⁵ PFU of vABb1EG-irEG baculovirus. EGFP expression was visualized by fluorescence microscopy as a function of time over several days as shown. (D) Tissue tropism of baculovirus in adult A. aegypti. A. aegypti adult mosquitoes were microinjected intrathoracically with 1×10⁵PFU of vABblEG-irEG baculovirus and observed at 15 days post-transduction. EGFP fluorescence was observed in head, antennae, proboscis, leg, wing, haltere, midgut malpigian tubules, ovary, crop and fat body tissues of adult mosquitoes.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

For convenience, certain terms employed in the specification, examples and appended claims are collected here. Unless defined otherwise, all technical and scientific terms used herein have the same meaning as commonly understood by one of the ordinary skill in the art to which this invention belongs.

Provided herein are recombinant baculoviruses, and methods for transducing mosquitos by use of the present recombinant baculoviruses.

### 1. Definitions

The term “baculoviruses” as used herein refer to arthropod-specific, double stranded DNA viruses that can be used to control insect pests (e.g., mosquitos). The nuclear polyhedrosis viruses (“NPV”) are one baculovirus subgroup. Various baculoviruses, including those that infect cotton bollworm, Helicoverpa zea, tobacco budworm, Heliothis virescens, Douglas fir tussock moth, Orygia pseudotsugata, gypsy moth, Lymantria dispar, alfalfa looper, Autographa californica, European pine sawfly, Neodiiprion sertifer, and codling moth, Cydia pomonella, are suitable as the vectors for expressing exogenous proteins in the infected hosts. In general, baculoviruses with wide host range are preferred, such as Autographa californica multiple nucleopolyhedrovirus (AcMNPV). Examples of baculovirus suitable for use in the present invention include, but are not limited to, AcMNPV, Anagrapha falclfera MNPV (AfMNPV), Anticarsia gemmatalis MNPV (AgMNPV), Bombyx mori MNPV (BmMNPV), Buzura suppressaria single nucleopolyhedrovirus (BsSNPV), Helicoverpa armigera SNPV (HaSNPV), Helicoverpa zea SNPV (HzSNPV), Lymantria dispar MNPV (LdMNPV), Orgyia pseudotsugata MNPV (OpMNPV), Spodoptera frugiperda MNPV (SfMNPV), Spodoptera exigua MNPV (SeMNPV), and Trichoplusia ni MNPVMNPV).

The phase “a gene encodes or encoding” refers to a nucleic acid which contains sequence information for a structural RNA such as rRNA, a tRNA, or the primary amino acid sequence of a specific protein or peptide. This term specifically encompasses degenerate codons (i.e., different codons which encode a single amino acid) of the native sequence or sequences which may be introduced to conform with codon preference in a specific host cell.

The term “an exogenous gene” generally denotes a nucleic acid that has been isolated, cloned and ligated to a nucleic acid with which it is not combined in nature, and/or introduced into and/or expressed in a cell or cellular environment other than the cell or cellular environment in which said nucleic acid or protein may typically be found in nature. The term encompasses both nucleic adds originally obtained from a different organism or cell type than the cell type in which it is expressed, and also nucleic acids that are obtained from the same cell line as the cell line in which it is expressed.

The term “recombinant” as a modifier of a vector (i.e., a nucleic acid), such as recombinant viral vectors, as well as a modifier of sequences such as recombinant polynucleotides and polypeptides, means that the compositions have been manipulated (i.e., engineered) in a fashion that generally does not occur in nature. A particular example of a recombinant vector, such as a recombinant baculoviral vector would be where a polynucleotide that is not normally present in the wild-type viral genome is inserted within the viral genome.

As used herein, the term “operably linked” refers to the linkage of nucleic acid fragments in such a functional relationship that when expressed, each of them can operate without functional problems, whereas, when one neucleic acid fragment is connected to the other, its function and expression may be affected by the other. For instance, the term refers to a functional linkage between a nucleic acid sequence coding for the desired protein and a nucleic acid expression control sequence in such a manner as to allow general functions. The operable linkage may be prepared using a genetic recombinant technique that is well known in the art, and site-specific DNA cleavage and ligation may be carried out using enzymes that are generally known in the art.

The term “transduce” refers to introduction of a nucleic acid into a cell or host organism by way of a vector (e.g.; the recombinant baculoviral vector of the present disclosure). Introduction of a transgene into a cell by a recombinant baculovirus is therefore be referred to as “transduction” of the cell. The transgene may or may not be integrated into genomic nucleic acid of a transduced cell (e.g., sf12 cells and mosquito C6/36 cells). If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently.

The term “transfection” as used herein refers to the process of introducing nucleic acids into a host cell, without the use of a virus or viral particle carrier. In other words, nucleic acids are introduced into a host cell via use of an agent which, when added to a complex of nucleic acid and transfection lipid in suitable quantities, is sufficient to overcome the deleterious effect of serum in the culture medium on transfection. Transfection agent may be polycations such as polybrene and are commercially available. An effective amount of a transfection agent is that quantity which produces a measurable increase in transfection efficiency for a given host cell cultured in the presence of serum.

The phase “a reporter protein” refers to a protein that is engineered to be expressed along with a protein-of-interest and induces visually identifiable characteristics usually involve fluorescent and luminescent proteins. For example, the gene that encodes jellyfish green fluorescent protein (GFP), which causes cells that express it to glow green under blue light, or the enzyme luciferase, which catalyzes a reaction with luciferin to produce light. Common examples of reporter protein include, but are not limited to, GFP, Discosoma sp. red fluorescent protein (DsRed), enhanced green fluorescence protein (EGFP), blue fluorescence protein (BFP), enhanced yellow fluorescent proteins (EYFP), Anemonia majano fluorescent protein (amFP), Zoanthus fluorescent protein (zFP), Discosoma fluorescent protein (dsFP), and Clavularia fluorescent protein (cFP).

The singular forms “a,” “and,” and “the” are used herein to include plural referents unless the context clearly dictates otherwise. Other than in the operating examples, or where otherwise indicated, all numbers expressing quantities of ingredients, reaction conditions, and so forth used in this application are to be understood as being modified in all instances by the term “about.” Accordingly, unless the contrary is indicated, the numerical parameters set forth in this application are approximations that may vary depending upon the desired properties sought to be obtained by the present invention.

### 2. The Recombinant Baculovirus

The present disclosure aims at providing a recombinant baculovirus for introducing exogenous DNA into a pest insect, particularly, mosquitos that transmit diseases.

To identify promoter(s) suitable for the purpose of this invention, expression gene cassettes carrying a reporter protein are independently constructed and linked to the candidate promoter sequence, which includes pag1, a HzNV-1 viral early expressing gene; p10, baculovirus late gene; cmv, cytomegalovirus; sv40, simian virus 40; lir, chimeric internal ribosome entry site (IRES) of RhPV virus and EV71 virus; b1, cecropin b1 gene; a4, defensin a4 gene; pub, polyubiquitin gene; and heat shock protein 70 (hp70) gene; so that a baculoviral transfer vector is produced. The transfer vector is then used with the baculoviral DNA to co-transfect a host cell (e.g., a mosquito cell line C6/36), and the capability of each candidate promoter sequences is evaluated by the expression of the reporter protein. Among the candidate promoter sequences that were tested, pag1, ceropin b1 gene and hp70 gene are relatively strong promoters that significantly drive the expression of the operably linked genes, whereas the rest of the promoters respectively exhibit mild to negligible activities.

Accordingly, the first aspect of the present disclosure is directed to a recombinant baculovirus, which is characterized in having a HzNV-1 viral early expressing gene pag1, a ceropin gene b1, a defensin a4 gene or a hp70 gene as a promotor for driving the expression of a foreign gene operably linked thereto.

To construct the present recombinant baculovirus, expression gene cassettes carrying an exogenous gene of interest are independently constructed and linked to the promoter sequence of pag1, ceropin b1, defensin a4 or hp70 gene to produce a transfer vector. The transfer vector is then used with the baculoviral DNA to co-transfect a host cell to produce the recombinant baculovirus.

According to preferred embodiments of the present disclosure, the baculoviral transfer vector is co-transfected with a Bac-N-Blue viral DNA into an insect host cell. The Bac-N-Blue viral DNA provides the necessary viral backbone, which contains the propagation-essential genes. Homologous recombination between the recombinant baculoviral transfer vector and the Bac-N-Blue viral DNA in the insect host cell allows the generation of a recombinant baculovirus, which is capable of propagating in the insect host cell and thereby producing the exogenous proteins of interest respectively encoded by the expression gene cassettes. The recombinant baculovirus was further selected and purified, such as by following the expression of a reporter polypeptide. Suitable insect host cell that may be used in the present disclosure includes, but is not limited to, S. furgiperda IPBL-9 (Sf9) cell, Sf21 cell, High Five cell, Minic Sf9 cell, and C6/C36 cells. According to some embodiments of the present disclosure, the insect host cell is Sf21 cell. According to other embodiments of the present disclosure, the insect host cell is C6/C36 cell. According to embodiments of the present disclosure, the transduction does not significantly affect the viability of the host insect cells for up to 12 days.

Optionally, reporter polypeptides are included in the baculoviral vectors. Examples of reporter polypeptide include, but are not limited to, blue fluorescence protein (BFP), cyan fluorescence protein (CFP), Discosoma sp. red fluorescent protein (DsRed), green fluorescence protein (GFP), enhanced green fluorescence protein (EGFP), enhanced yellow fluorescence protein (EYFP), and etc. In some preferred embodiments of the present disclosure, the reporter polypeptide is EGFP. It should be noted that the reporter polypeptide (e.g., EGFP) is not a necessary feature for the aim of this invention, which is, introducing exogenous genes of interest into a pest insect (e.g., mosquitos).

### 3. Use of the Present Recombinant Baculovirus

The recombinant baculovirus constructed in accordance with the methods described above will carry at least one exogenous gene of interest, whose expression is driven by the promoter sequence of pag1, ceropin b1, defensin a4, or hp70. The thus produced recombinant baculovirus may then be used to transduce pest insects, such as mosquitos of Aedes aegypti, A. albopictus, Culex tritaeniorhynchus, and Anopheles sinensis

Accordingly, the present disclosure also encompasses a method of introducing an exogenous gene into a mosquito. The method includes the step of, transducing the mosquito with the present recombinant baculovirus without suppressing the production of microRN (miRNAs) therein.

Upon viral infection, antiviral defense mechanisms within the infected host are activated to fight off the infected virus. The antiviral defense mechanism includes activation of one or more proteins in the immunity system, for example, activation of Dosha, Dicer, Exportin-5, Toll-like receptor-2 (TLR-2), STAT1, STATE, interleukin 7R, interleukin 1A, and etc. It has been suggested that to infect a cell with a baculoviral vector, agents that suppress the expression of proteins in the antiviral defense pathway, such as agents that suppress the expression of Dosha, Dicer, Exportin-5, Toll-like receptor-2 (TLR-2), STAT1, STATE, interleukin 7R, and interleukin 1A, are preferably administered prior to, or currently with, the baculoviral vector (see US 2013/0109077A1). Such agents include but are not limited to, agents that activate the production of miRNAs that cleave the mRNAs of proteins in the antiviral defense pathway.

Unlike the teaching provided in US 2013/0109077A1, it was unexpectedly discover that infecting a pest insect with the present recombinant baculoviruses does not require use of agents to suppress the antiviral defense pathway in the pest insect. According to preferred embodiments of the present disclosure, mosquitos may be transduced with the exogenous gene of interest by the present recombinant baculoviruses without using agents that suppress the expression of Dosha, Dicer, Exportin-5, Toll-like receptor-2 (TLR-2), STAT1, STATE, interleukin 7R, and interleukin 1A in the mosquitos.

According to embodiments of the present disclosure, the entry of the present recombinant baculovirues into mosquitos is achieved by GP64 envelop protein.

According to embodiments of the present disclosure, the present recombinant baculovirus can transduce strong gene expressions in both larvae and adults of mosquitos in different genus, which include but are not limited to, Aedes aegypti, A. albopictus, Culex tritaeniorhynchus, and Anopheles sinensis. Preferably, the present recombinant baculovirus is used to transduce mosquitos of Aedes aegypti or A. albopictus.

According to embodiments of the present disclosure, the present recombinant baculovirus does not replicate in the infected mosquitos.

According to embodiments of the present disclosure, the exogenous gene transduced by the present recombinant baculovirues are expressed in mosquito tissues that include, but are not limited to, head, proboscis, leg, haltere, midgut malphigian tubules, ovary, crop, and fat body of the mosquito.

The following examples are provided to illustrate the present invention without, however, limiting the same thereto.

### EXAMPLES

Material and Methods

Cells and Viruses.

A. albopictus C6/36 and A. aegypti CCL-125 cell clones were maintained in RPMI-1640 medium (Invitrogen) supplemented with 10% fetal bovine serum (FBS), 2 mM L-glutamine, 0.1 mM non-essential amino acids, 1 mM sodium pyruvate, and 2% penicillin-streptomycin at 28° C. in a 5% CO₂ humidified incubator. The Spodoptera frugiperda IPLB-Sf21 (Sf21) cells were grown at 26° C. in TC100 insect medium containing 10% FBS. HEK-293T and Vero-E6 cells were maintained in Dulbecco's modified Eagle's medium (DMEM; Gibco) supplemented with 10% FBS and 2% penicillin-streptomycin at 37° C. in a 5% CO₂ humidified incubator. The Autographa californica nucleopolyhedrovirus (AcMNPV) baculovirus (gene bank accession number NC_001623.1) genome (flashback-ultra) was used to generate recombinant baculoviruses in this study. Viral frozen stocks in respective culture mediums were stored at −80° C.

Construction of Transfer Vectors and Recombinant Baculoviruses.

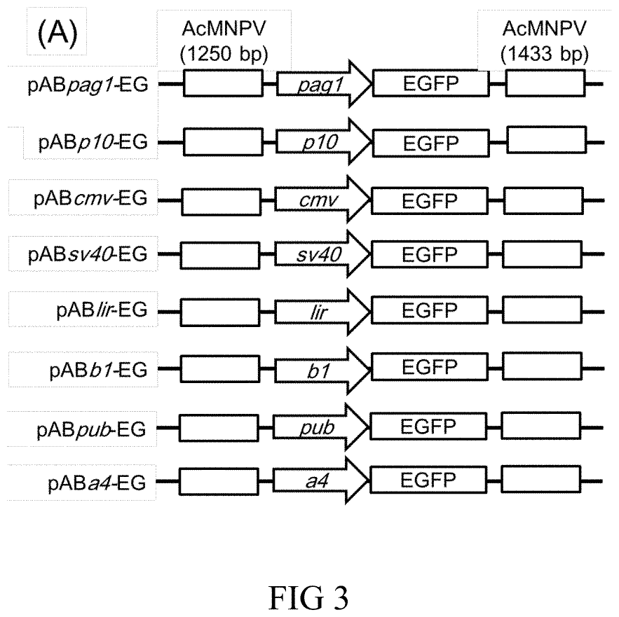

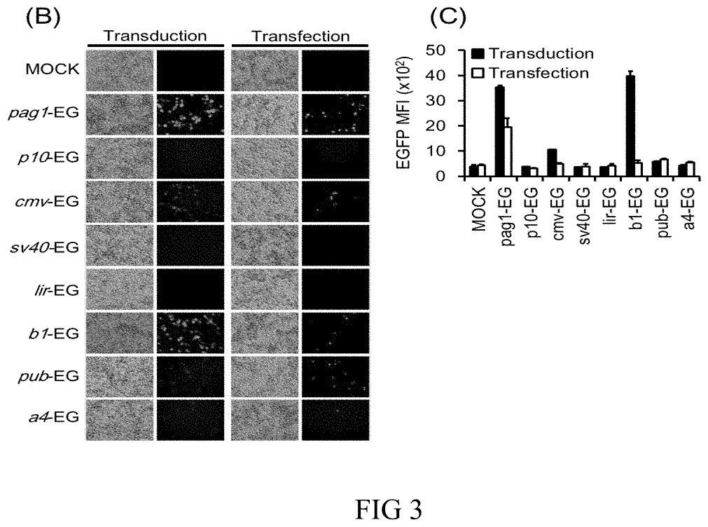

The transfer vector pABspmC was constructed to generate the vABspmC recombinant baculovirus. Mammalian sv40 and HzNV-1 pag1 gene promoters were inserted upstream of the TriEX promoter in the reverse orientation in pTriEx-3 vector (Novagen). The PCR-amplified mCherry gene from pJET-mCherry vector was subcloned downstream of the pag1 promoter to generate the final transfer vector, named pABspmC. In this composite vector, mCherry expression is driven by the sv40 and pag1 promoters in the mammalian and insect systems, respectively. For promoter analysis, we constructed several transfer vectors as listed in FIG. 3, panel A. The PCR-amplified promoter regions were cloned upstream of EGFP in the pABhEG plasmid (Abvector), replacing the hsp70 promoter. The promoter regions of pag1, baculovirus early gene; p10, baculovirus late gene; cmv, cytomegalovirus; sv40, simian virus 40; lir, chimeric internal ribosome entry site (IRES) of RhPV virus and EV71 virus; as well as two mosquito genes Cecropin b1 and Defensin a4 (GenBank IDs: HQ285957.1, and HQ285959.1) both amplified by PCR from the A. aegypti genome; and the commercially-synthesized Polyubiquitin gene (GenBank ID: GU179018) promoter region (565 bp upstream of Pub gene) (named respectively as the pABpag1EG, pABp10EG, pABcmvEG, pABsv40EG, pABlirEG, pABb1EG, pABa4EG, and pABpubEG transfer vectors). These transfer vectors were constructed by following the standard protocols of the In-Fusion Cloning Kit (Clontech). The transfer vector pBacb1EG-irEG was constructed according to traditional cloning methods. Briefly, a NcoI-XbaI EGFP fragment was subcloned into the NcoI and XbaI sites of pGL3 to create the plasmid, pGL3-EGFP. Both NheI-BglII promoter fragments of Cecropin b1 were amplified and subcloned into pGL3-EGFP to create pGL3-b1EGFP. The restriction enzyme-digested region between NheI-NotI from pGL3-b1-EGFP was subcloned into pBac-CHIKV-26S-Rhir-E (Wu Y. J., (2008) Acta Pharmacologica Sinica 29(8):965-974) to replace the CHIKV-26S region and the large NheI-BglII fragment of pBac-b1-EGFP-Rhir-E was inserted and self-ligated to create the basic vector. All the above transfer vectors were verified by sequencing. Briefly, these transfer vectors were co-transfected with vAcRP23.Laz (Pharmingen), a linearized viral DNA of AcMNPV, into Sf21 cells by using Cellfectin (Life Technologies). The resulting recombinant baculoviruses were isolated through end-point dilutions, amplifications in T75 flasks, and virus titers were determined by TCID50 analysis in Sf21 cells.

Baculovirus Transduction and Infection.

Mosquito C6/36 cells or mammalian cells were plated at a density of 4×10⁵ cells or 1×10⁵ cells (unless stated) per well in 24-well plates 1 day before transduction. The cells were washed with 1×DPBS and baculovirus was added at different MOIs as indicated in respective medium without any FBS or antibiotics. Plates were centrifuged at 2000 rpm for 32 min at room temperature (RT). Transduced mosquito or mammalian cells were incubated at 28° C. or RT, respectively, for 2 h. The plates were washed twice with DPBS containing 0.1% trypsin to remove unattached viruses. Then complete 10%-FBS medium was added and cultured for various time-points as indicated. For baculovirus infection assays, Sf21 cells were plated at a density of 2×10⁵ cells per well in 24-well plates 1 day before infection and baculovirus was added at different MOIs. The plates were centrifuged at 2000 rpm for 32 min at RT and then incubated at 26° C. for various timeframes as indicated.

Cell Proliferation and Cell Viability Analyses.

Mosquito C6/36 cells were transduced with recombinant vABspmC baculovirus at an MOI=1, 10 and 100 for 4 days. Cells were harvested by trypsinization every 24 h post-transduction, resuspended in 1×DPBS, and then mixed with equal volumes of 0.4% trypan blue exclusion dye (Sigma) to exclude dead cells and determine healthy cell proliferation. For cell viability analysis, relative cell metabolic activity was determined by adding 10% v/v AlamarBlue to the cells and incubating for 4 h. Reductions in AlamarBlue concentrations were measured with a fluorescence reader (EnSpire, PerkinElmer) at an excitation wavelength of 560 nm and an emission wavelength of 590 nm.

Baculovirus Neutralization Assay.

The vABspmC baculovirus (4×10⁵PFU) at MOI=1 was treated with 1 μg of neutralizing (AcV1, Santa Cruz Biotechnology) or non-neutralizing monoclonal antibodies (AcV5, Santa Cruz Biotechnology) against GP64 protein in a total volume of 300 μl with 1×DPBS and centrifuged at 300 rpm for 1 h at RT. The virus+antibody mixture was then added to C6/36 cells for 2 h and kept at 28° C. in a 5% CO₂ humidified incubator. The virus+antibody mixture was then removed and washed three times with 1×DPBS containing 0.1% trypsin to remove unattached virus particles. Complete 10%-FBS medium was then added, and the cells were cultured for 2 days before examining mCherry fluorescence.

Plasmid DNA Transfection.

Mosquito C6/36 cells were plated at a density of 4×10⁵ cells per well in 24-well plates. The next day, recombinant plasmid DNA (0.5 μg in FIG. 3) was mixed with transfection reagent (TransIT-Insect Transfection Reagent; Minis) as per manufacturer recommendations and incubated for 30 min at RT, before the DNA+transfection reagent mixture was added to the cells. After 5 h in a 28° C. incubator, the cells were removed and washed with 1×DPBS, before 10%-FBS complete medium was added. The cells were then incubated at 28° C. for 2 days before being collected for flow cytometry analyses.

Preparation of Cells for Flow Cytometry Analyses.

Mosquito C6/36 cells were transduced with baculovirus in 24-well plates for various designated timeframes. The medium was removed from the wells, cells were washed twice with 1×DPBS, and then treated with 100 μl of pre-warmed trypsin-EDTA for 5 min. Then 100 μl of MEM (modified Eagle's medium; Gibco) was added to neutralize the trypsin-EDTA and cells were collected in 1 ml of 1×DPBS in 1.5 ml Eppendorf tubes. The cells were centrifuged at 5000 rpm for 5 min to remove the MEM and the cell pellet was washed at least three times with 1×DPBS. Finally, cells were resuspended in 1 ml of fixing buffer (1% FBS+1% formaldehyde diluted in 1×DPBS) and analyzed by flow cytometry. The numbers of mCherry- or EGFP-positive cells and the mean fluorescence intensities (MFI) were determined using flow cytometry and FlowJo software.

Quantification of Baculovirus DNA by Real-Time qPCR.

Baculovirus-infected Sf21 or baculovirus-transduced C6/36 cells were extensively washed three-times with 1×DPBS containing 0.1% trypsin to remove free virus particles from cells. Total cellular DNA was extracted using the High Pure PCR Template Preparation Kit (Roche). The quantity and quality of DNA was checked using a NanoDrop ND-1000 spectrophotometer (Thermo Fisher Scientific). Real time-qPCR was performed in a 96-well plate, with each well containing 2 μl DNA (100 ng/μl), 0.6 μl 10 μM specific primers (final concentration, 300 nM), 6.8 μl H₂O, and 10 μl 2×SYBR Green Master Mix (ABI). Samples were run in triplicate. Real-time PCR was conducted in the ABI 7500 FAST system for over 40 cycles with an annealing temperature of 60° C. Assessment of the expression of each target gene was based on relative quantification (RQ) using the comparative critical threshold (CT) value method. The RQ of a specific gene was evaluated in each reaction by normalization to the CT obtained for the endogenous control genes (Actin gene in C6/36 cells and GAPDH in Sf21 cells). Three independent transfection experiments were conducted, and the data was the results from one independent infection experiment.

Mosquito Rearing.

The A. aegypti (Kaoshiung strain), A. albopictus (Chung-Ho strain), C. tritaeniorhynchus (Peitou strain), and Anopheles sinensis were reared at 27° C. and at 80% humidity under a 12-h light/dark cycle. Larval stages were fed on pulverized fish food, and adults were provided with 5% sucrose ad libitum.

Mosquito Virus Oral Infection and Intrathoracic Inoculation.

Frozen stocks of viruses were thawed at 37° C., and four or five 10-fold serial dilutions of virus were made in RPMI1640 medium, which were then mixed with an equal volume of defibrinated sheep blood. Each viral dilution was presented to 4-5-day post-eclosion female A. aegypti or A. albopictus mosquitoes (starved for 24 h). Cold-anesthetized mosquitoes were intrathoracically inoculated with virus according to the protocol of described by Thompson and Sarnow ((2003) Virology 315(1): 259-266).

### Example 1 Baculovirus Transduction into Mosquito C6/36 Cells

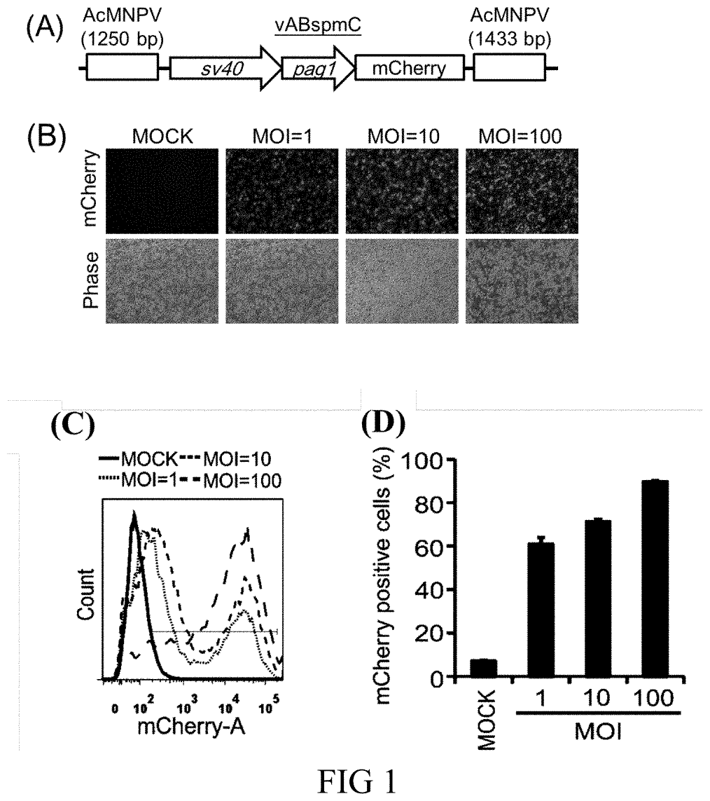

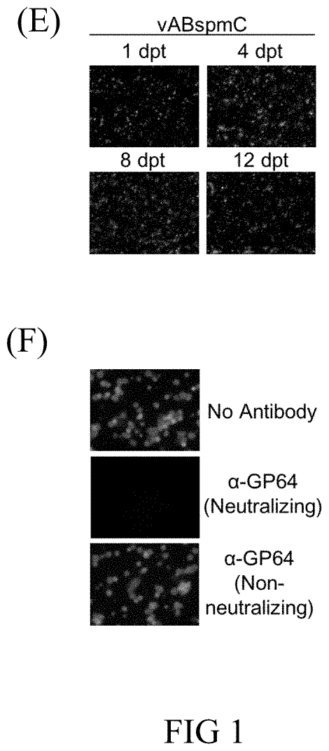

In this example, the use of AcMNPV for delivering gene expression cassette in mosquito host C6/36 cells was investigated. To this purpose, recombinant vABspmC baculovirus vector containing sv40 and pag1 promoters for driving the expression of mCherry gene was constructed (FIG. 1, panel A), and C6/36 cells were respectively transduced by the recombinant vector at MOI=1, 10 and 100. The fluorescence of mCherry was observed as early as 12 h (data not shown), and significant dose-dependent fluorescence intensity was detected at 48 h post-transduction (FIG. 1, panel B).

Transduced cells were then collected and analyzed by flow cytometry to determine transduction efficiency. Gating of mCherry-positive cells and quantification revealed that transduction efficiencies of 60%-65% (FIG. 1, panel D) could be routinely achieved at MOI=1, and efficiency increased to 95%-100% at MOI=100 (FIG. 1, panels C and 1D).

To investigate whether C6/36 cells undergo a lytic cycle after virus infection and suffer a reduction in cell proliferation, C6/36 cells were exposed to the vABspmC virus at various MOIs for several timeframes, then stained with 0.4% trypan blue solution to exclude dead cells and the numbers of healthy cells were counted. Cell duplication was examined every 24 h up to 96 h post-transduction. It was found that normal cell proliferation continued until 48 h post-transduction for all MOI's, with a mild decrease in cell propagation at later time-points, as compared to mock conditions, especially for MOI=100 (data not shown). Cell viability was analyzed at 4-day intervals by adding 10% v/v AlamarBlue to determine cell metabolic activity. No apparent toxicities were found in C6/36 cells at MOI=1 and MOI=10 for all exposure timeframes, however, a mild but prominent reduction was observed for MOI=100 at 12 days post-transduction (data not shown).

Whether C6/36 cells supported persistent gene expression was also investigated, and the toxicity of baculovirus on C6/36 cells was monitored for up to 12 days. As illustrated in FIG. 1, panel E, mCherry expression increased as a function of time up to 8 days post-exposure before reaching saturation. Thus, the result suggested that this expression system did not seem to involve the baculovirus lytic cycle, and C6/36 cells could sustain baculovirus transduction for long periods.

As baculovirus enters insect or mammalian cells through surface GP64 proteins, accordingly, GP64-mediated cell entry was also determined, in which baculovirus was treated with neutralizing or non-neutralizing GP64 antibodies before being transduced into C6/36 cells. It was found that baculovirus treated with GP64-neutralizing antibody did not show mCherry fluorescence at 48 h post-transduction compared to a negative control (FIG. 1, panel F), which indicated that baculovirus indeed entered mosquito hosts through the GP64 surface protein.

Taken together, results in this example suggest that baculovirus could successfully enter and persistently drived transgene expression in mosquito C6/36 cells without affecting cell proliferation of the host cells.

### Example 2 Replication Analysis of Baculovirus in Mosquito C6/36 Cells

In this example, replication of baculovirus in mosquito C6/36 cells was investigated via monitoring the accumulated viral DNA level in the host cells. To this end, the natural host of baculoviru-insect Sf21 cells were included as a control, and were infected with vABspmC along with C6/36 cells.

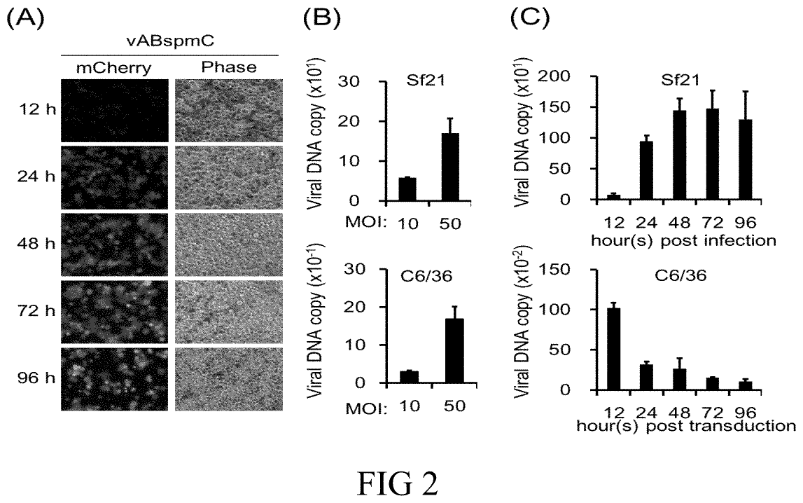

The mCherry fluorescence time-course images of C6/36 cells transduced with vABspmC at MOI=10 are provided in FIG. 2, panel A; and the baculovirus entry efficiency in both Sf21 cells and C6/36 cells at MOI=10 and MOI=50 are provided in the bar graphs of FIG. 2, panel B. Relative amounts of intracellular viral DNA at respective MOIs 2h post-infection/transduction were found to be similar in both hosts, suggesting that baculovirus entered C6/36 and Sf21 cells via similar efficiency (FIG. 2B).

Further, viral DNA accumulation in sf21 cells increased gradually with time from 12 h to 48 h post-infection, and reached a plateau level 96 h post-infection. By contrast, viral DNA in C6/36 cells decreased gradually with time from 12 h to 96 h post-transduction, suggesting that baculovirus likely did not replicate in mosquito C6/36 cells (FIG. 2, panel C).

### Example 3 Functional Analysis of Various Baculovirus-Incorporated Promoters and their Efficiencies in C6/36 Cells

In this example, the functionality and efficiency of various promoters were tested by inserting their DNA sequences into the baculovirus genome. To this purpose, transfer vectors with different promoter sequences driving EGFP expression were constructed, and depicted in FIG. 3, panel A.

After packaging each transfer vector into baculovirus, C6/36 cells were transduced at MOI=1 so that the majority of cells contained one or fewer viral genomic copies per cell. For comparison, C6/36 cells were also transfected with 500 ng plasmid of the same transfer vector so that each cell was transfected with an average of 1.15×10⁵ copies of plasmid DNA. EGFP images were taken at 48 h post-transduction (FIG. 3, panel B). The strength of each promoter in C6/36 cells was evaluated by quantifying mean fluorescence intensities of EGFP-positive cells by flow cytometry. The quantified results are provided in FIG. 3, panel C.

It was found that pag1 and b1 promoters are consistently stronger among others, and b1 driven EGFP expression was slightly better than pag1 driven EGFP expression. By contrast, the p10, sv40, lir, pub and a4 promoters exhibited little or negligible fluorescence, and the cmv promoter only showed minimal activity.

In the plasmid transfection system, pag1 promoter showed better activity while the activity of other promoters remained basal or below the detection limit. Furthermore, under plasmid transfection, the p10, sv40, lir, and a4 promoters exhibited similar results to baculovirus transduction (i.e., no fluorescence), and the cmv promoter only presented basal activity. It is quite surprising that the pub promoter consistently showed less fluorescence in the baculovirus transduction system but remained functional in the plasmid transfection system. We speculated that it might be due to the baculovirus-mediated cellular response suppressing pub promoter efficiency.

### Example 4 Functional Analysis of Heat Shock Protein Promoter and its Efficiency in C6/36 Cells

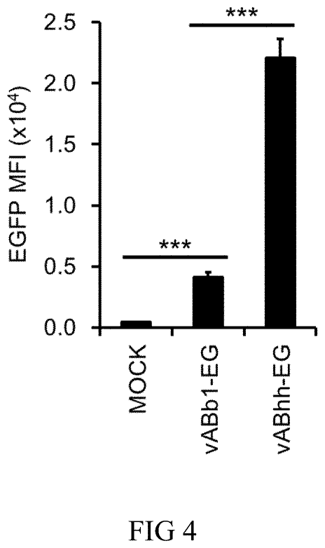

In this example, transfer vectors respectively containing b1 promoter and heat shock protein 70 (hp70) were constructed, and subsequently used to transduce C6/36 cells in accordance with similar procedures as described in Example 2, in which the promoter activities were determined by flow cytometry. Results is illustrated in FIG. 4.

Compared to the mock control, b1 promoter was capable of driving EGFP expression in C6/36 cells; however, hp70 exhibited surprisingly strong promoter activity, thus is the strongest promoter for baculovirus mediated transgene expression.

### Example 5 Baculovirus-Mediated Gene Expression in Larvae and Adults of Various Mosquito Species

In this example, the transduction ability of baculovirus into larvae of different mosquito species was investigated. Aedes aegypti (the yellow fever mosquito) and A. albopictus (Asian tiger mosquito or forest mosquito) are responsible for the transmission of various potentially fatal viruses in humans including dengue, chikungunya, zika, yellow fever, and Mayaro, as well as several filarial nematodes such as Dirofilaria immitis and other diseases. Culex (Culex) tritaeniorhynchus is the main vector of Japanese encephalitis. Anopheles sinensis transmits malaria and lymphatic filariasis.

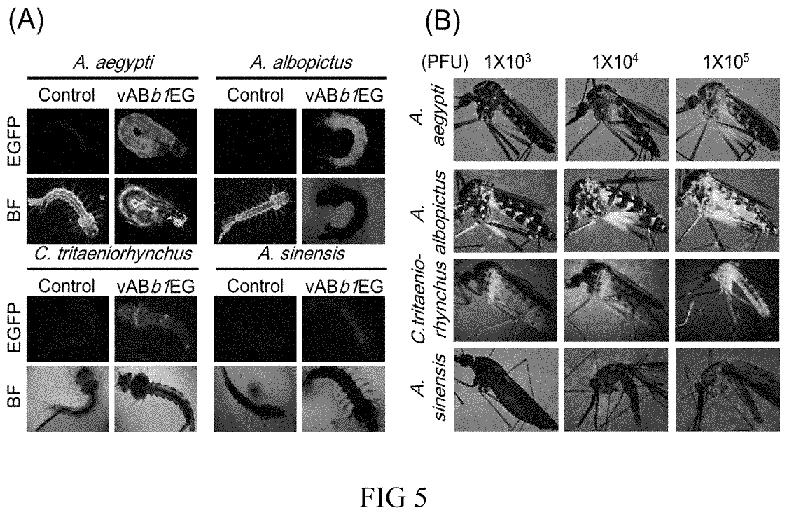

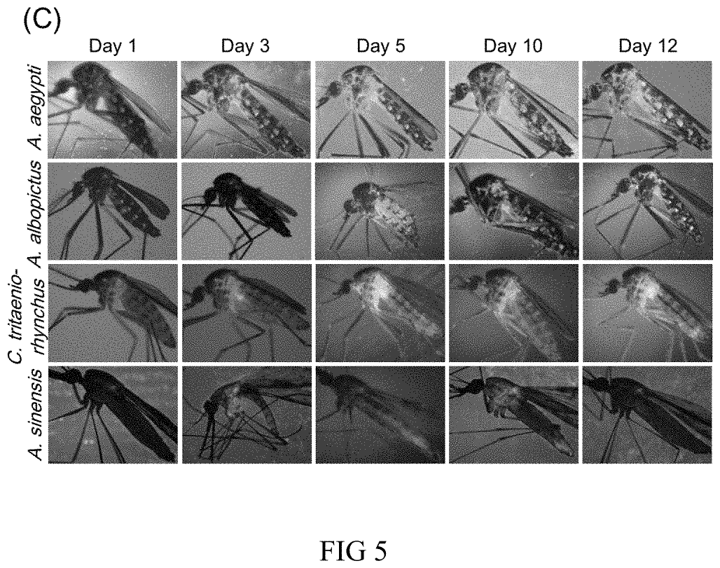

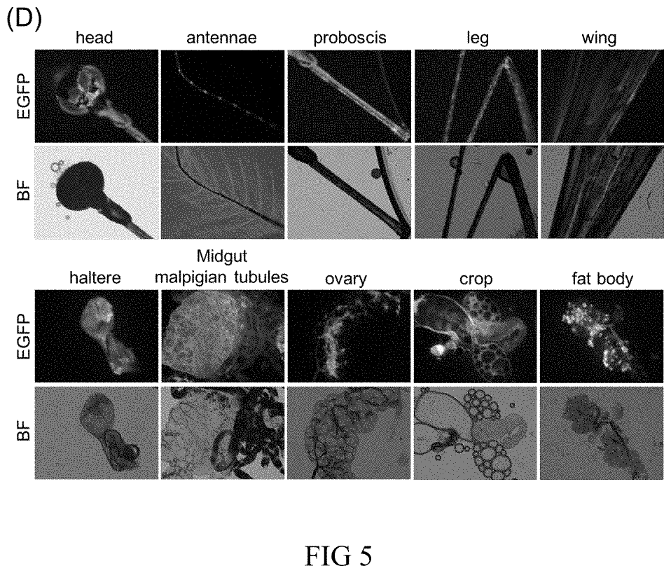

To this purpose, a dose of 1×10⁵ PFU recombinant vABb1EG-irEG baculovirus was microinjected into larvae of the afore-indicated four mosquito species and EGFP expression was measured 2 days post-transduction. Results are provided in FIG. 5, in which strong EGFP expressions were found in A. aegypti and A. albopictus, by contrast, the EGFP expressions in C. tritaeniorhynchus and A. sinensis were weak (FIG. 5, panel A).

To investigate the transduction ability of baculovirus into adult mosquitoes, the same recombinant baculovirus (vABb1EG-irEG) was dose-dependently microinjected intrathoracically into larvae of the afore-indicated four mosquito species. Interestingly, baculovirus-mediated EGFP expression in adult mosquitoes was higher with increasing viral dosages at 6 days post-microinjection. Again, weaker EGFP expression in A. sinensis was observed, as compared to other three mosquito species (FIG. 5, panel B). Further, RT-qPCR on total genomic DNA isolated from transduced adult mosquitoes was performed so as to determine whether baculovirus could replicate in vivo. Results indicated that baculovirus did not replicate in adult mosquitoes (data not shown), which corroborated the finding in in vitro experiments.

Next, microinjected mosquitoes were examined at regular time-points through a pre-designated timeframe. It was found that EGFP expression was weak at 1 day post-injection, but increased thereafter throughout the experiment until 12 days post-injection (FIG. 5, panel C). To determine the tissue tropism of baculovirus-mediated foreign gene expression, the transduced mosquitoes were dissected. EGFP expression was strongly observed in most of the tissues including head, proboscis, leg, haltere, midgut malphigian tubules, ovary, crop, and fat body, but somewhat weak expression was observed in the antennae and wings (FIG. 5, panel D).

Taken together, these results suggest that baculovirus can successfully transduce and express transgenes in the larvae and adults of the mosquito species tested here with little or no tissue barriers.

It will be understood that the above description of embodiments is given by way of example only and that various modifications may be made by those with ordinary skill in the art. The above specification, examples, and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A recombinant baculovirus capable of delivering an exogenous gene into a mosquito comprising a promoter; and the exogenous gene operably linked to the promoter.

2. The recombinant baculovirus of claim 1, wherein the promoter is any of a HzNV-1 viral early expressing gene pag1, a ceropin gene b1, a defensin gene a4, or a gene of heat shock protein 70 (hp70).

3. The recombinant baculovirus of claim 2, further comprising a reporter gene encoding a reporter protein that is any of green fluorescence protein (GFP), enhanced green fluorescence protein (EGFP), Discosoma sp. red fluorescent protein (DsRed), blue fluorescence protein (BFP), enhanced yellow fluorescent proteins (EYFP), Anemonia majano fluorescent protein (amFP), Zoanthus fluorescent protein (zFP), Discosoma fluorescent protein (dsFP), or Clavularia fluorescent protein (cFP).

4. The recombinant baculovirus of claim 2, wherein the baculovirus is any of Autograph californica multiple nucleopolyhedrovirus (AcMNPV), Anagrapha falclfera MNPV (AfMNPV), Anticarsia gemmatalis MNPV (AgMNPV), Bombyx mori MNPV (BmMNPV), Buzura suppressaria single nucleopolyhedrovirus (BsSNPV), Helicoverpa armigera SNPV (HaSNPV), Helicoverpa zea SNPV (HzSNPV), Lymantria dispar MNPV (LdMNPV), Orgyia pseudotsugata MNPV (OpMNPV), Spodoptera frugiperda MNPV (SfMNPV), Spodoptera exigua MNPV (SeMNPV), or Trichoplusia ni MNPVMNPV).

5. The recombinant baculovirus of claim 4, wherein the baculovirus is AcMNPV.

6. A method of introducing an exogenous gene into a mosquito comprising transducing the mosquito with a recombinant baculovirus without suppressing the production of microRNAs (miRNAs) in the mosquito, wherein the recombinant baculovirus comprises a promoter; and the exogenous gene operably linked to the promoter.

7. The method of claim 6, wherein the promotor is any of a HzNV-1 viral early expressing gene pag1, a ceropin gene b1, a defensin gene a4 or hp70.

8. The method of claim 7, wherein the suppressing of the production of miRNAs in the mosquito is achieved by use of an agent that leads to suppressing the expression of Drosha, Dicer, Toll-like receptor 2, STAT1, STAT6, interleukin 7R, or interleukin 1A.

9. The method of claim 6, wherein the mosquito is Aedes aegypti or A. albopictus.

10. The method of claim 9, wherein the mosquito is a cell, a larvae or an adult.

11. The method of claim 6, wherein the baculovirus is any of Autographa californica multiple nucleopolyhedrovirus (AcMNPV), Anagrapha falclfera MNPV (AfMNPV), Anticarsia gemmatalis MNPV (AgMNPV), Bombyx mori MNPV (BmMNPV), Buzura suppressaria single nucleopolyhedrovirus (BsSNPV), Helicoverpa armigera SNPV (HaSNPV), Helicoverpa zea SNPV (HzSNPV), Lymantria dispar MNPV (LdMNPV), Orgyia pseudotsugata MNPV (OpMNPV), Spodoptera frugiperda MNPV (SfMNPV), Spodoptera exigua MNPV (SeMNPV), or Trichoplusia ni MNPVMNPV).

12. The method of claim 11, wherein the baculovirus is AcMNPV.

13. The method of claim 7, wherein the recombinant baculovirus further comprises a reporter gene encoding a reporter protein that is any of green fluorescence protein (GFP), enhanced green fluorescence protein (EGFP), Discosoma sp. red fluorescent protein (DsRed), blue fluorescence protein (BFP), enhanced yellow fluorescent proteins (EYFP), Anemonia majano fluorescent protein (amFP), Zoanthus fluorescent protein (zFP), Discosoma fluorescent protein (dsFP), or Clavularia fluorescent protein (cFP).

14. The method of claim 7, wherein the recombinant baculovrus does not replicate in the mosquito.
